(19) United States
(12) Patent Application Publication
(10) Pub. No.: US 2010/0168632 A1
(43) Pub. Date: Jul. 1, 2010

(54) Bunion reliever and method of treatment

(76) Inventors: Soraya Ann Abbassian, Boring, OR (US); Ted Robert Skipper, Boring, OR (US)

Correspondence Address: MARK S. HUBERT P.C., 516 SE MORRISON- SUITE 1200, PORTLAND, OR, 97214, US

(21) Appl. No.: 12/319,026

(22) Filed: Dec. 31, 2008

(51) Int. Cl. A61F 5/00; A61F 13/06; A61B 19/00

(52) U.S. Cl. 602/30; 128/894; 128/893; 128/898

(57) ABSTRACT

A bunion reliever apparatus utilizes a rigid, concave, and tapered brace to place counter-pressure directly on the bunion. Pain relief is immediate as the big toe is urged back into its natural alignment. A stabilizing strap secures the brace along the side of the foot and a correction strap encircles and is tightened around the big toe. The correction strap forces the outside of the big toe to contact the rigid brace. Since the force securing the stabilizing strap to the foot exceeds the force exerted on the big toe by the correction strap, a counter-force is transferred directly onto the most exaggerated extension of the bunion by the rigid brace. For use in conjunction with the bunion reliever apparatus a set of exercises for relaxation, re-direction, and new tissue memory is provided.

### BACKGROUND OF THE INVENTION

Bunions are boney growths or enlargements of the large joint below the big toe. A bunion grows in response to faulty mechanics of the first metatarsophalangeal joint (the joint between the big toe and the foot). Almost all bunions are related to the combination of a faulty gait worsened by ill fitting shoes that put pressure higher up the foot (towards the end of the toes) than the metatarsophalangeal joint. The result of that force causes the first metatarsophalangeal joint to deform, enlarge, and grow bone tissue to help balance gait. This cycle of pressure and the joint's response to that pressure produces the bunion, usually over the course of many years. Once a bunion has formed, the mechanics of the foot and toes are further altered, exacerbating the problem. Tendons begin to pull the big toe into an abnormal position, angulated towards the other toes and the base of the first metatarsophalangeal joint becomes more and more prominent. The bunion continues to worsen in part because of the new angulation of the toe, and in part due to inflammatory changes over the bunion surface.

Bunions frequently accompany problems such as hammer toes, corns, calluses, and ingrown toe nails, with their associated infections. In addition the blood supply changes with the enlarging joint. If left untreated, the bunion may become so painful that the patient begins walking differently to relieve the pressure. As the problem progresses, pain may become so severe that walking at all becomes intolerable. In extreme situations surgery is recommended to alleviate the pain and deformity associated with the bunion. However, surgery is painful, costly, and often, over time, the bunion develops again, because surgery simply does not address the underlying cause of the faulty mechanics.

Previous bunion relief products all avoid placing pressure on the bunion itself. Many of these products attempt to protect and cushion the bunion in some form or another. The prevalent theory in the prior art is to minimize pain and any unwanted effect on blood supply to the bunion. However, it is this dogma that the present invention completely opposes. When pressure is not placed on the bunion itself, the mechanical problem cannot truly improve. No other splint or supporter places counter-pressure on the bunion to relieve pain and stop the further growth of bunions. Over time, depending on how severe the bunion is, the present invention can improve the pain and malformation.

Henceforth, such a bunion reliever apparatus and method of treatment with the described advantages would fulfill a long felt need in the medical industry. This new invention utilizes and combines known and new technologies in a unique and novel configuration to overcome the aforementioned problems and accomplish this.

### SUMMARY OF THE INVENTION

The general purpose of the present invention, which will be described subsequently in greater detail, is to provide an apparatus, and method of treatment for the non-surgical correction of a bunion.

It has many of the advantages mentioned heretofore and many novel features that result in a new bunion reliever apparatus and method of treatment which is not anticipated, rendered obvious, suggested, or even implied by any of the prior art, either alone or in any combination thereof.

In accordance with the invention, an object of the present invention is to provide an improved apparatus, and method of treatment for the non-surgical correction of a bunion that applies direct pressure to the inflamed first metatarsophalangeal joint.

It is another object of this invention to provide an improved apparatus, and method of treatment for the non-surgical correction of a bunion that allows the patient the ability to heat and cool the apparatus for added pain relief.

It is a further object of this invention to provide an improved apparatus, and method of treatment for the non-surgical correction of a bunion that allows the patient to insert a hot or cold pack with the apparatus.

It is still a further object of this invention an improved apparatus, and method of treatment for the non-surgical correction of a bunion to that is easy to manufacture and inexpensive to purchase and easy to employ.

It is yet a further object of this invention to provide an improved apparatus, and method of treatment for the immediate pain relief associated with a bunion.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements. Other objects, features and aspects of the present invention are discussed in greater detail below.

### BRIEF DESCRIPTION OF THE DRAWINGS

FIG. 1 is a top view of the bones in a normal right foot;

FIG. 2 is a top view of the bones in a right foot in the beginning stages of the formation of a bunion;

FIG. 3 is a top view of the bones in a right foot afflicted with a bunion and illustrating bone growth secondary to mechanicl irritation;

FIG. 4 is a top view of a right foot inflicted with a bunion growth secondary to mechanicl irritation;

FIG. 5 is a top view of the present invention in the right foot configuration;

FIG. 6 is a bottom view of the present invention in the right foot configuration;

FIG. 7 is a perspective view of the rigid elongated member;

FIG. 8 is a top view of the bunion reliever apparatus the left foot configuration;

FIG. 9 is an end view of the rigid elongated member (loop portion of a hook-and-loop fastener omitted);

FIG. 10 is a cross-sectional view of the rigid elongated member in an alternative embodiment (loop portion of a hook-and-loop fastener omitted); and

FIG. 11 is a partial top view of a foot with the present invention engaging a bunion afflicted foot.

### DETAILED DESCRIPTION

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

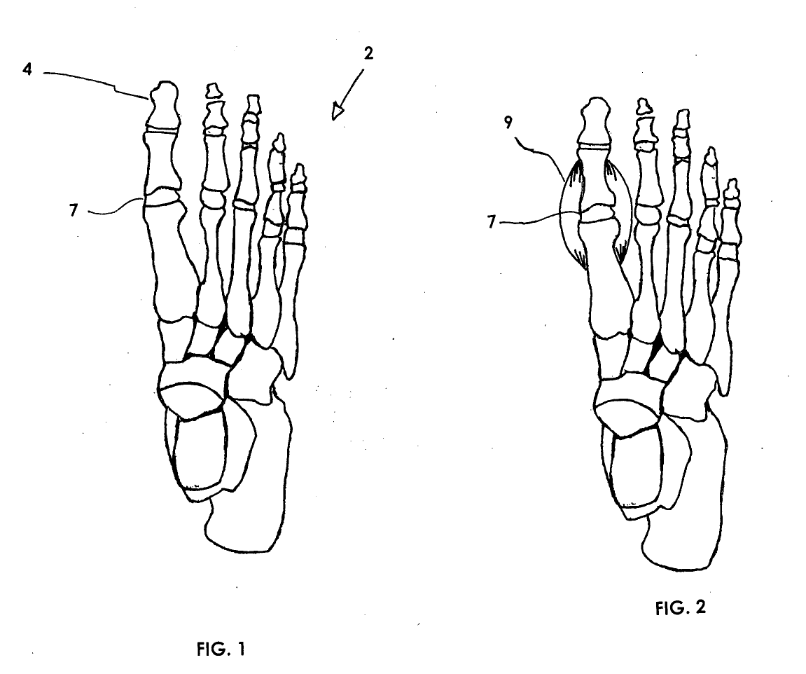

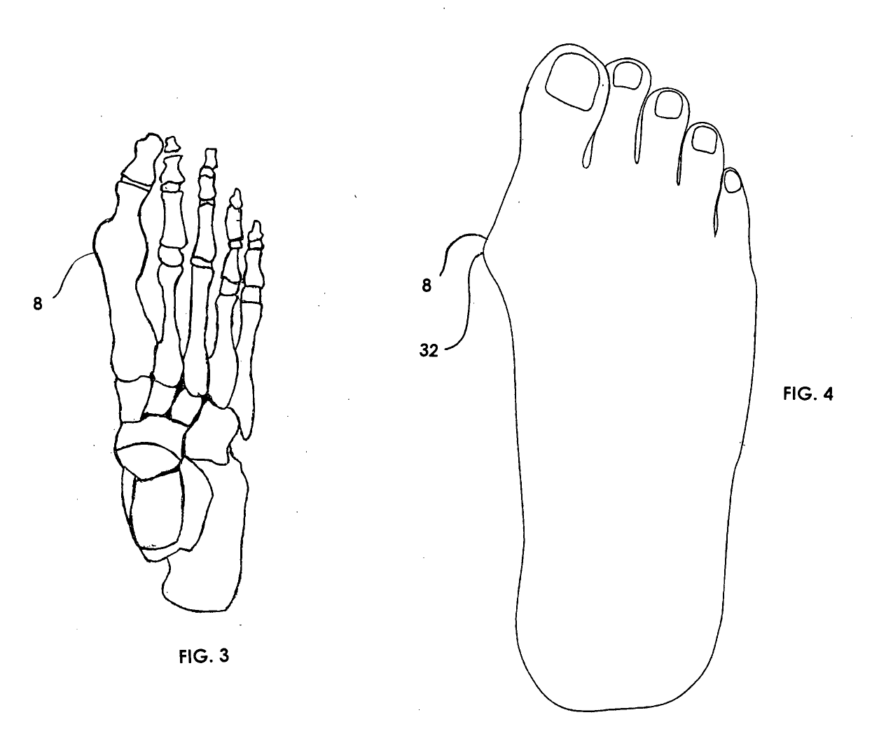

Referring now to FIGS. 1-4 the progressive misalignment of the first metatarsophalangeal joint 7, which starts with a slight leaning of the big toe towards the second toe, and then gradually produces the characteristic bump, can be seen. In FIG. 1 the bone structure of a non-bunion afflicted foot 2 is visible, illustrating how the big toe 4 resides parallel to the longitudinal axis of the foot. Over time, due to a biomechanical abnormality, the area surrounding the first metatarsophalangeal joint 7 becomes inflamed 9 as seen in FIG. 2. If mechanical irritation continues, bunion formation progresses as micro-fractures and abnormal boney growth around the first metatarsophalangeal joint 7. The body produces new bone growth (bunion) 8 in attempt to heal the micro-fractures as is visible in FIGS. 3 and 4.

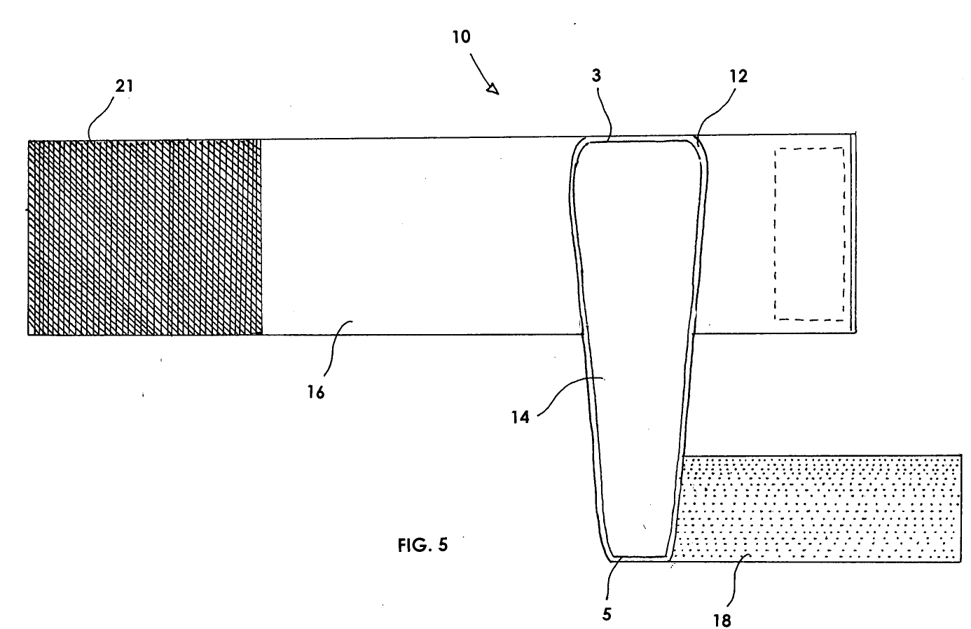

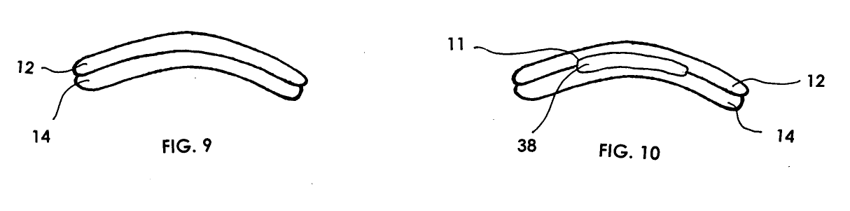

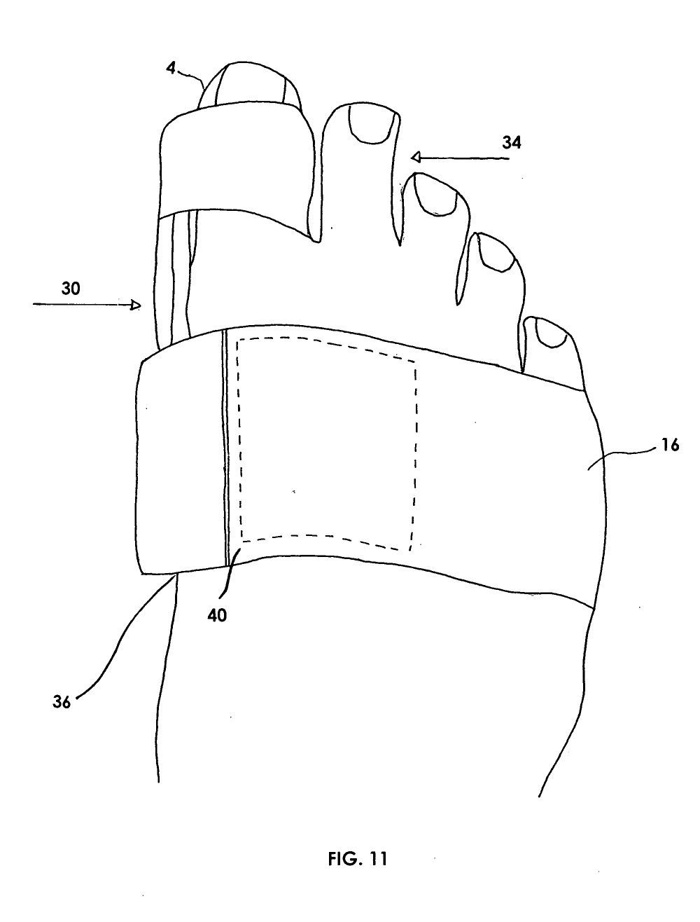

A first embodiment of the present invention is illustrated in FIGS. 5-9. The bunion reliever apparatus 10 is comprised a rigid elongated brace 12, comfort padding 14, a stabilizing strap 16, and a correction strap 18.

Brace 12 extends between stabilizing strap 16 and correction strap 18. Brace 12 has three critical structural differences setting it apart from all the prior art. These structural differences are dictated by the device's theory of operation. Brace 12 is longitudinally tapered, concave in axial cross-section, and completely inflexible or rigid both longitudinally and axially. Functionally, brace 12 is designed to apply a first force directly perpendicular to the longitudinal axis of the foot 2, as indicated by arrow 30, at the inside of the big toe, and apply a second force parallel to the first force at the farthest most extension of the bunion 32 but in an opposite direction as indicated by arrow 34. It is to be noted that arrows 30 and 34 represent force vectors of the same magnitude in opposing directions. In providing these forces brace 12 cannot flex along its longitudinal axis, must contact as much of the outer surface of the bunion 8 as possible, and must align and maintain itself on an axis residing in a plane approximately parallel with the longitudinal axis of the foot 2, established at the arch 36.

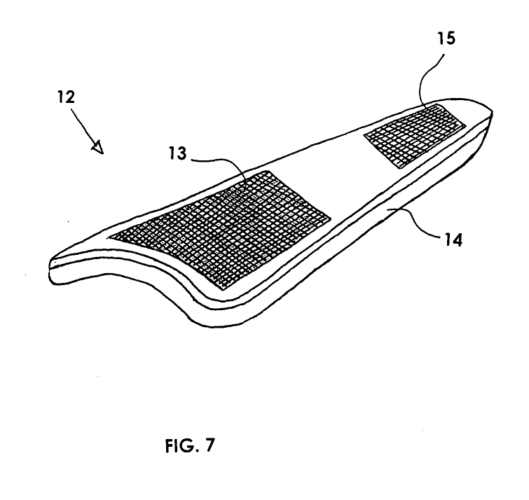

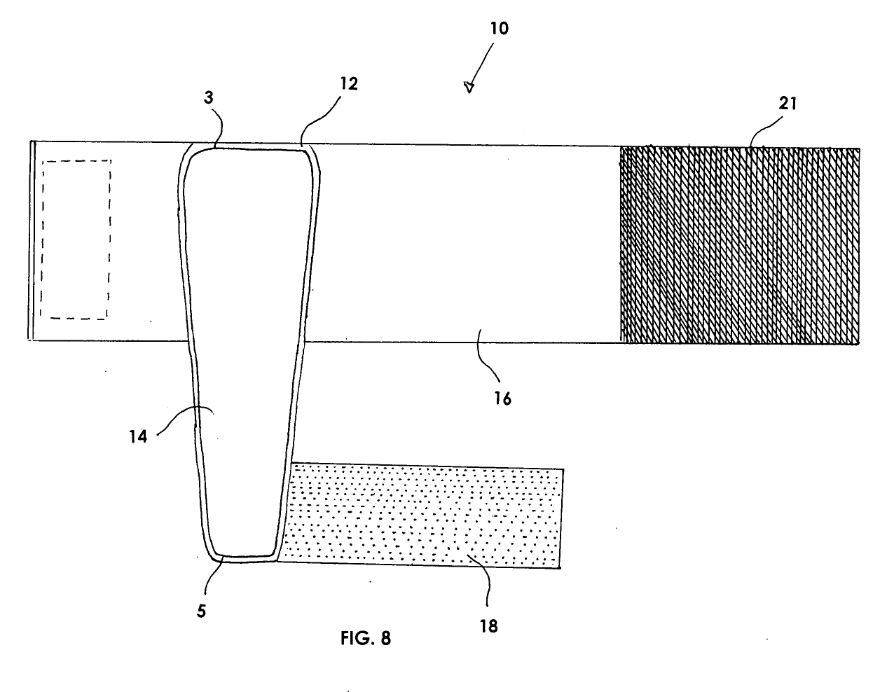

The thickness and material of construction for brace 12 imparts the rigidity that prevents brace 12 from flexing about bunion 8 so as ensure the force vectors 30 and 34 are properly maintained. The concavity of brace 12 applies the force vector 30 evenly and with a maximized surface area on the bunion 8. This minimizes pressure sores on the bunion 8. The concavity in conjunction with the longitudinal taper ensures that the brace is properly aligned with natural downward taper of the foot 2 and minimizes slippage of the brace 12. The concavity and taper of brace 12, simply does more than cradle the upper, inner (arched) side of the foot 2, but rather is an integral part of the theory and operation of the present invention. FIG. 7 illustrates the concavity and taper of brace 12. All edges on brace 12 are contoured for comfort.

While several materials would be suitable for the construction of the brace 12, the present invention employs an acetal polymer which exhibits enhanced stiffness, dimensional stability, impact resistance, and structural strength, similar to steels. The polymer selected for brace 12 may retain heat and cold as to enable the user to externally heat or cool bunion reliever apparatus 10 prior to donning. Also visible in FIG. 7 is the comfort padding 14, which resides along the concave surface of brace 12. Comfort padding 14 aides in maintaining proper placement and securement of brace 12 along the foot by virtue of its high coefficient of friction with the skin of the foot. It provides resistance for the brace 12 to slide down the foot towards the big toe due to the forces exerted by the stabilizing strap 16 and correction strap 18. It also provides a more comfortable “feel” to the user.

In the preferred embodiment ¼ inch, closed cell foam is used for the comfort padding 14. ¼ inch, closed cell foam form fits to the user's foot, which decreases the pain initially experienced as the big toe 4 undergoes realignment. The closed cell foam of this thickness actually conforms to the user's foot allowing for greater variance in the length of time the bunion reliever apparatus 10 can be worn, while decreasing the possibility of vascular compression or pressure ulcers. The apparatus 10 actually becomes unique to each person's bunion 8. The closed cell feature minimizes the potential for bacterial buildup and simplifies washing. In one embodiment the comfort padding has one outer surface with a self adhesive facing applied thereto so as to be removable and or replaceable. This feature is critical when switching the apparatus 10 between different feet, as each bunion has its own discrete physical configuration.

In a first alternate embodiment visible in FIG. 10, an orifice 11 resides between brace 12 and comfort padding 14, such that a freezable or heatable fluid pack 38 can be inserted to increase the therapeutic effects. In the preferred embodiment, the overall length of brace 12 is 3 and ⅞ inches and the thickness of the polymer is approximately ⅛ of an inch. The narrow end 5 of brace 12 is 0.500-0.563 inches wide, and the wide end 3 is approximately 1.000-1.150 inches wide. The taper of the brace is 6.6^{•}±1^{•}. The outside diameter of wide end 3 of brace 12 is 1.700-2.000 inches, while the inside diameter of wide end 3 is 1.720-1.750 inches. The outside diameter of narrow end 5 is 1.075-1.150 inches, and the inside diameter of narrow end 5 is 0.825-0.855 inches. It is to be noticed that because the location of the forces to be transmitted through the brace 12 are paramount, the fit of the brace along the foot is critical. For this reason, the concavity of the brace 12 along the linear axis is not constant, rather the concavity increases going from the wide end 3 to the narrow end 5. This has been shown to garner the best fit. Stabilizing strap 16 is mechanically or chemically affixed between its proximate and distal end to the convex surface of brace 12 adjacent the wide end 3 of brace 12.

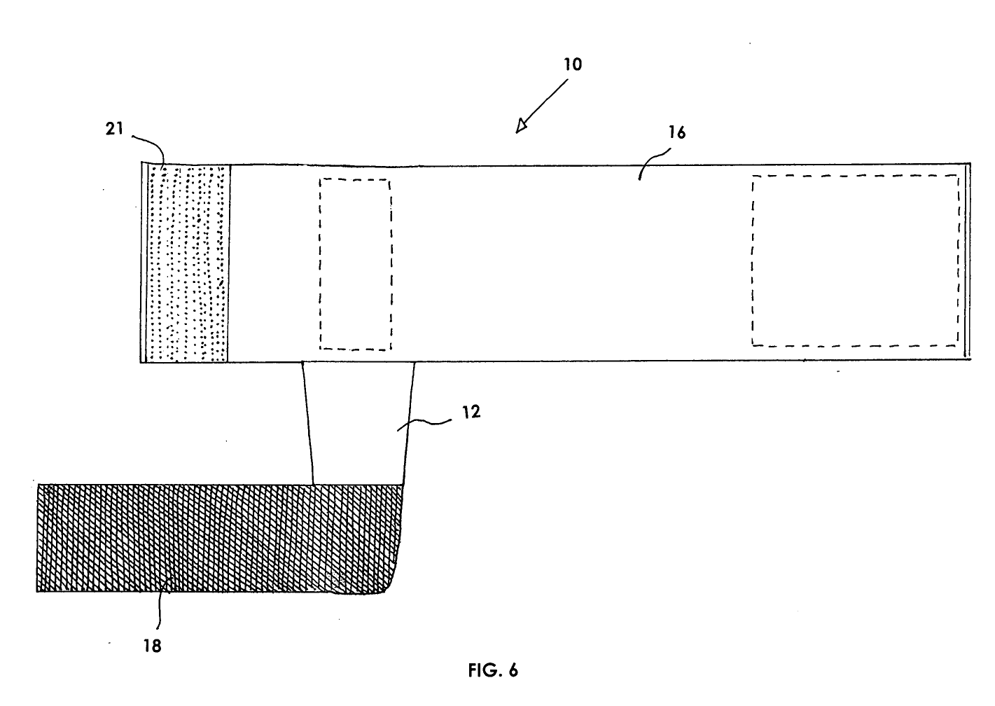

In the preferred embodiment stabilizing strap 16 has matingly engageable hook-and-loop interlocking fastener portions 21 on opposite sides of the distal and proximate ends. There is also a first loop portion 13 of a hook-and-loop fastener for mechanically affixing stabilizing strap 16, and a second loop portion 15 of a hook-and-loop fastener for mechanically affixing correction strap 18 to brace 12 which is visible in FIG. 7. Stabilizing strap 16 includes a matingly engageable hook portion 21 of an hook-and-loop interlocking fastener for engagement with first loop portion 13 on brace 12. Employing a hook-and-loop fastener on the convex surface of brace 12 allows for the brace 12 to be removed from stabilizing strap 16 rotated 180° and reattached to stabilizing strap 16. This allows for a right and left foot configuration wherein the doubled over section 40 of stabilizing strap 16 resides on the top of the foot rather than on the bottom. This allows comfortable walking without misalignment of the foot's bone structure. FIG. 8 illustrates bunion reliever apparatus 10 in the left foot configuration, and FIGS. 5 and 6 illustrate the bunion reliever apparatus 10 in the right foot configuration. However, alternative methods of fastening could comprise any number of mechanical fasteners such as hooks, buttons, dome snap assemblies, or other mechanical fasteners as is well known in the art. The advantage of the using hook-and loop fasteners 18 is the capability of accommodating various foot sizes without adding the weight of snap or buckle assemblies. In the preferred embodiment, stabilizing strap 16 is approximately 2 inches wide and is constructed from approximately 70% polyester and 30% rubber similar to an elastic bandage, which is well know in the art. It should be noted that the percentage of polyester and rubber can vary considerably and still produce the desired result, as manufactures often alter the percentages of rubber and polyester.

Correction strap 18, is similarly affixed to brace 12 along narrow end 5. Unlike stabilizing strap 16, correction strap 18 is not elastic, is only 1 inch wide, and instead of hook-and-loop fastening sections, correction strap 18 is comprised of a self-looking, bundling material, wherein opposite sides of correction strap 18 adhere to one another; that is, one entire side of correction strap 18 is comprised of loops, while the opposite side is comprised of hooks. The non-elastic feature is a critical feature of the apparatus 10. Movement of the foot, such as may be encountered when walking, would cause the big toe to move slightly with respect to the brace 12 and continually shift the bones in the foot affecting the overall healing alignment that the apparatus strives to maintain. The correction strap 18 may be directly affixed to the brace 12 or it may be engaged with second loop fastener portion 15 such that it may be adjusted when the apparatus 10 is being adjusted for use on a different foot. It is to be noted that this removal and reattachment of correction strap 18 is not critical to the morphing between the left foot and right foot configuration.

Stabilizing strap 16 and correction 18 are extremely stout and the hook-and-loop fasteners occupy considerable surface area on each strap as the forces required to urge the foot proper alignment are considerable. In the preferred embodiment the stabilizing strap 16 is approximately 2 inches wide and 11 inches long, and the correcting strap 18, is one inch wide and 5 and ½ inches long.

The theory of operation is as follows: stabilizing strap 16 secures non-flexible brace 12 along the side of the foot 2 with the foot's natural downward taper. Correcting strap 18 encircles and is tightened around big toe 4 with first force 30 such that the outside of the big toe firmly contacts comfort padding 14 on the narrow end 5 of brace 12 as much as each person's bunion allows. Since the force securing brace 12 to the foot 2 exceeds first force 30, the wide end 3 of brace 12 is constrained firmly against the side of foot 2 and does not break contact with the surface of the foot. Thus, a second force 34 (counter force) is transferred directly onto the most exaggerated extension of the bunion 8 concurrent with the application of first force 30 long the entire inner side of the big toe 4. Since the force (pressure) exerted by stabilizing strap 16 is larger than the first force 30, rigid brace 12 cannot rock or tilt about the bunion 8. This causes the bunion 8 and the toe 4 to be equally forced back into their natural alignment as much as each person's bunion will allow. The source of the misalignment is adjusted by a balanced force on opposing sides. The vectors of these forces are equal in magnitude but opposite in direction. Stated otherwise, the amount of force exerted to align the big toe 4 is directly transferred to the center of the bunion for its alignment. This first force 30 moves the big toe 4 into natural alignment and the second force 34 spreads out all the adjacent toe knuckles, making relieving spaces that treat the entire foot's malalignment. Pain relief is immediate as the big toe 4 is urged back into proper alignment.

It is the rigidity, concavity, and taper of the brace 12 that efficiently transmits the highest amount of counter force onto the bunion 8.

It should be noted that straightening is not the first goal of the bunion relief apparatus 2. Initially, the apparatus 2 is just for the relief of pain. In time relief of the bunion can occur too. Experimentation has shown that it is best to wear the brace every night for 15 minutes, although it can be worn more than once during the day for 15 minute episodes with 2 hour intervals if so desired.

To optimize the use of the bunion reliever apparatus 2, foot exercises and physical therapy are recommended. If one spends a little time with these exercises before donning the brace, relief will be more rapid and permanent. Alternating heat and ice can also improve and relieve bunion pain and soft tissue swelling. The brace 12 can tolerate external heat and cold applied for improved response and correction. The following set of bunion brace physical therapy exercises utilized in conjunction with the bunion reliever apparatus 2 has proven to be the method of treatment found to elicit the best results for pain relief, bunion minimization, and possible correction of foot malalignment. This method of use prepares the foot for the apparatus 2 through three mechanisms. First, these techniques promote the relaxation of muscles, ligaments, and joints. The relaxation removes tissue memory from the abnormal gait and bunion formation movement. Second, the re-direction exercises require the exact opposite motions and directions of gait to invoke a change of memory pattern in the tissues. Finally, exercises to elicit new tissue memory openness for the application of apparatus 2 are provided. The following method of performing exercises is performed before donning the bunion relief apparatus 2 for a 15 min interval daily.

Relaxation exercises:
1). Massage the leg from the calf muscle to the foot, squeezing the Achilles tendon. Massage the foot and each toe by stroking along each metatarsal to each toe tip in a linear fashion away from the body.
2). Contract and relax the calf muscle (3 times), contract and relax the ankle flexors and extensors (3 times), and pull the toes towards the nose then grippingly curl the toes, (3 times).

### Re-direction Exercises:

1). Squeeze then twist the calf muscle in thirds along its length.
2). Squeeze and circle massage the tissue on the shin bone in thirds along its length.
3). Rotate the ankle three times outwardly and three times inwardly with the ankle in the jutted out position. Point the toes and circle with the foot and ankle clockwise and counter-clockwise three times each.
4). Pull the big toe straight, and directly forward in alignment with the axis of the foot, and hold this traction-position for three seconds. Repeat this traction pull on the rest of the toes Repeat three times.

### New Tissue Openness Exercises:

1). Squeeze heel with (and in) the palm of the hand, firmly, for 3 seconds. Repeat three times.
2). Bend knee such that the foot bottom is now face up and the body of the foot is cradled in two hands. Massage the arch of the foot with both thumbs in linear strokes along the linear axis of the foot for approximately 30 seconds.
3). Press foot tissue firmly at the base of small toes and hold three seconds alternating with toe traction. (This may be optionally done with the four toes simultaneously, treating these toes as a group.)
4). Press the foot tissue firmly at base of the big toe, hold three seconds, pull the big toe into traction, hold, and then rotate in three circles while in traction. Repeat three times.

With practice, these exercises can be completed very quickly. Then the bunion relief apparatus 2 can be applied in 15 min intervals, as is desired by the user throughout the day. Should these exercises be employed in conjunction with the bunion relief apparatus 2 from the very beginning stages of bunion formation, surgery will be avoided and overall foot health will be maintained.

The above description will enable any person skilled in the art to make and use this invention. It also sets forth the best modes for carrying out this invention. There are numerous variations and modifications thereof that will also remain readily apparent to others skilled in the art, now that the general principles of the present invention have been disclosed. As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Having thus described the invention, what is claimed as new and desired to be secured by Letters Patent is as follows:

1. A bunion reliever apparatus comprising:
a rigid, tapered, linear member, wherein said rigid linear member has a non-uniform convex outer face, a non-uniform concave inner face, a wide distal end, a narrow proximate end;
an elastic stabilizing strap affixed at said wide distal end; and
a non-elastic correction strap affixed at said narrow end;
wherein said linear member tapers along a longitudinal axis to accommodate the natural taper of the foot, and said stabilizing strap is adapted for encircling engagement of the foot in an orientation approximately perpendicular to said linear member, and said correction strap is adapted for encircling engagement of a big toe in an orientation approximately perpendicular to said linear member, and when said stabilizing strap and said correction strap are engaged said linear member applies a linear counter-force directly on the bunion urging said big toe into its natural position.

2. The apparatus of claim 1 wherein said convex outer face has a convexity that is lesser at the wide distal end then at the narrow proximate end, and said concave inner face has a concavity that is lesser at the wide distal end then at the narrow proximate end.

3. The apparatus of claim 2 further comprising non-slip comfort padding affixed to said concave side of said linear member.

4. The apparatus of claim 3 wherein said stabilizing strap has a first end and a second end with engageably conformed hook-and-loop fasteners operatively affixed thereto to forcefully and adjustably secure said linear member to said foot.

5. The apparatus of claim 4 wherein said correcting strap has a third end and a fourth end with engageably conformed hook-and-loop fasteners operatively affixed thereto to forcefully and adjustably secure said linear member to said big toe.

6. The apparatus of claim 5 wherein said linear member is comprised of a material capable of retaining heat or cold.

7. The apparatus of claim 5 wherein an orifice resides between said linear member and said comfort padding that a freezable or heatable fluid pack can be inserted.

8. The apparatus of claim 6 wherein said linear member further comprises one portion of a hook-and-loop fastener affixed to said convex side of said linear member and wherein said stabilizing strap further comprises a matingly engageable portion of a hook-and-loop fastener between its distal and proximate ends to operatively and reversibly engage said convex side of said linear member.

9. A bunion reliever apparatus comprising:
a non-flexible, tapered, linear member with a wide distal end, a narrow proximate end, a convex outer face with a convexity that is lesser at the wide distal end then at the narrow proximate end, and a concave inner face with a concavity that is lesser at the wide distal end then at the narrow proximate end;
a stabilizing strap affixed at said wide distal end;
a correction strap affixed at said narrow end; and
a non-slip comfort padding affixed to said concave face;
wherein said linear member tapers along a longitudinal axis to accommodate the natural taper of the foot and has an increasing concavity of the inner face to accommodate the configuration of the foot, and said stabilizing strap is adapted for encircling engagement of the foot in an orientation approximately perpendicular to said linear member, and said correction strap is adapted for encircling engagement of a big toe in an orientation approximately perpendicular to said linear member, such that when said stabilizing strap and said correction strap are engaged said linear member applies a linear counter-force directly on the bunion urging said big toe into its natural position.

10. A method for pain relief and realigning of the first metatarsophalangeal joint of a bunion inflicted foot comprising the following steps:
1). Massage the leg from the calf muscle to the foot, squeezing the Achilles tendon. Massage the foot and each toe by stroking along each metatarsal to each toe tip in a linear fashion away from the body;
2). Contract and relax the calf muscle (3 times), contract and relax the ankle flexors and extensors (3 times), and pull the toes towards the nose then grippingly curl the toes, (3 times);
3). Squeeze then twist the calf muscle in thirds along its length;
4). Squeeze and circle massage the tissue on the shin bone in thirds along its length;
5). Rotate the ankle three times outwardly and three times inwardly with the ankle in the jutted out position. Point the toes and circle with the foot and ankle clockwise and counter-clockwise three times each;
6). Pull the big toe straight, and directly forward in alignment with the axis of the foot, and hold this traction-position for three seconds. Repeat this traction pull on the rest of the toes Repeat three times;
7). Squeeze heel with (and in) the palm of the hand, firmly, for 3 seconds. Repeat three times;
8). Bend knee such that the foot bottom is now face up and the body of the foot is cradled in two hands. Massage the arch of the foot with both thumbs in linear strokes along the linear axis of the foot for approximately 30 seconds;
9). Press foot tissue firmly at the base of small toes and hold three seconds alternating with toe traction. (This may be optionally done with the four toes simultaneously, treating these toes as a group.);
10). Press the foot tissue firmly at base of the big toe, hold three seconds, pull the big toe into traction, hold, and then rotate in three circles while in traction. Repeat three times;
11). Placing a rigid, tapered, linear member with a concave inner face along the bunion side of the bunion inflicted foot;
12.) Securing said rigid member to said side of foot via a stabilizing strap via encircling engagement of the foot in an orientation approximately perpendicular to said linear member;
13.) Further securing said rigid member to said side of foot via a correction strap via encircling engagement of the big toe in an orientation approximately perpendicular to said linear member;
14.) Tightening said stabilizing strap as to firmly secure said linear member to said side of foot;
15.) Tightening said correction strap about said big toe as to generate a linear counter-force directly on the bunion urging said big toe into its natural position.
16.) Remove said rigid member after approximately 15 minutes.

11. The apparatus of claim 9 wherein said linear member further comprises one portion of a hook-and-loop fastener affixed to said convex side of said linear member and wherein said stabilizing strap further comprises a matingly engageable portion of a hook-and-loop fastener between its distal and proximate ends to operatively and reversibly engage said convex side of said linear member.
